(19) United States
(12) Patent Application Publication
(10) Pub. No.: US 2026/0294296 A1
(43) Pub. Date: Oct. 1, 2026

(54) Improvements in or Relating to Capturing Biometric Data

(71) Applicant: Earswitch Ltd, Manchester (GB)

(72) Inventor: Nicholas GOMPERTZ, Manchester (GB)

(73) Assignee: Earswitch Ltd, Manchester (GB)

(21) Appl. No.: 19/480,612

(22) PCT Filed: May 3, 2024
(86) PCT No.: PCT/GB2024/051164

§ 371 Date: Oct. 31, 2025

(30) Foreign Application Priority Data
May 3, 2023 (GB) 2306488.4

(51) Int. Cl. A61B 5/1495; A61B 5/00; A61B 5/1455

(52) U.S. Cl. CPC A61B 5/1495; A61B 5/14552; A61B 5/6843; A61B 2503/40

(57) ABSTRACT

A method for capturing biometric data of a human and/or other animal, the method comprising: applying localised compression of biological tissue(s), so as to reduce blood volume in target biological tissue(s), and illuminating the target biological tissue(s); OR illuminating the target biological tissue(s) and applying localised compression of biological tissue(s), so as to reduce blood volume in target biological tissue(s); and detecting and analysing: one or more optical reflectance and/or transmission characteristics of the blood volume reduced target biological tissue(s); and/or one or more spectroscopy characteristics of the blood volume reduced target biological tissue(s).

### CROSS REFERENCE TO RELATED APPLICATIONS

This application is the U.S. National Phase Application of PCT International Application No. PCT/GB2024/051164, filed May 3, 2024, which claims priority to United Kingdom Patent Application No. 2306488.4, filed May 3, 2023, the contents of such applications being incorporated by reference herein.

### FIELD OF THE INVENTION

The present invention relates to a method and apparatus for capturing biometric data of a human and/or other animal. In particular, the invention relates improved detection and/or analysis of such biometric data and, thereby, improves oximetry and/or spectroscopic analysis.

### BACKGROUND OF THE INVENTION

It is understood that an individual's skin colour may detrimentally affect optically based analysis techniques conducted on the skin of that individual. For example oximetry, Raman and/or infrared spectroscopy can be affected by skin colour.

Skin colour is known to be linked to levels of melanin within the skin, and skin of a particularly dark tone often poses a challenge for capturing accurate biometric data. Existing techniques presently use a one-for-all type of analysis which cannot optimise detection for all skin colours/skin tones.

It is also understood that blood flow and oxygen saturation may detrimentally affect an objective measurement of skin colour and/or skin tone.

Further, it is understood that haemoglobin may obscure spectroscopic signals, masking the true molecular constituents of target biological tissue(s) and/or affecting their detection.

### SUMMARY OF THE INVENTION

The present invention is aimed at a method and apparatus for capturing biometric data which is unaffected by skin colour and/or skin tone. The present invention is also aimed at a method and apparatus for improving detection of spectroscopic signals—in particular, such detection being minus any adverse effects of haemoglobin absorption.

According to a first aspect, the present invention provides a method for capturing biometric data of a human and/or other animal, the method comprising:
applying localised compression of biological tissue(s), so as to reduce blood volume in target biological tissue(s), and illuminating the target biological tissue(s); OR
illuminating the target biological tissue(s) and applying localised compression of biological tissue(s), so as to reduce blood volume in target biological tissue(s); and
detecting and analysing:
   one or more optical reflectance and/or transmission characteristics of the blood volume reduced target biological tissue(s); and/or
   one or more spectroscopy characteristics of the blood volume reduced target biological tissue(s).

Preferably, the method further comprising detecting and analysing optical reflectance and/or transmission characteristics of non-blood volume reduced target biological tissue(s) either prior to applying localised compression or following reducing localised compression.

Preferably, detecting and analysing optical reflectance and/or transmission characteristics of the blood volume reduced and non-blood volume reduced target biological tissue(s) is conducted in either order.

Preferably, the method comprising reducing localised compression of biological tissue(s) to restore blood flow to target biological tissue(s), and detecting and analysing optical reflectance and/or transmission characteristics of the non-blood volume reduced target biological tissue(s).

Preferably, the method comprising detecting and analysing optical reflectance and/or transmission characteristics of blood volume reduced and non-blood volume reduced biological tissue(s) is conducted on the same target biological tissue(s).

Preferably, applying localised compression of biological tissue(s) impairs blood flow to target biological tissue(s) to reduce or prevent detection of an optical waveform/pulse signal.

Preferably, applying localised compression of biological tissue(s) provides at least a compressive pressure which: reduces or prevents blood flow to target biological tissue(s); compresses underlying blood vessels; provides a blanching effect to target biological tissue(s); and/or is equivalent to, or exceeds, a systolic blood pressure of an individual.

Preferably, wherein illuminating comprising:
ambient light; and/or
emitting one or more beams of light of one or more wavelengths.

Preferably, emitting the one or more beams of light comprising directly or indirectly illuminating the target biological tissue(s).

Preferably, the method comprising:
analysing one or more differences in the optical reflectance and/or transmission characteristics of the blood volume reduced target biological tissue(s) and non-blood volume reduced target biological tissue(s), and adjusting analysis of non-blood volume reduced target biological tissue(s) and/or applying one or more correction factors to analysis of non-blood volume reduced target biological tissue(s); and/or
utilising analysis of the optical reflectance and/or transmission characteristics of the blood volume reduced target biological tissue(s) to improve oximetry, Raman and/or IR spectroscopy (and/or other spectroscopy) of the target biological tissue(s).

Preferably, the method comprising applying localised compression to:
biological tissue(s) being different tissue(s) to the target biological tissue(s); or
biological tissue(s) being same tissue(s) as the target biological tissue(s).

Preferably, the blood volume reduced target biological tissue(s) is:
directly compressed biological tissue(s); or
non-compressed biological tissue(s).

Preferably, localised compression of biological tissue(s) temporarily impairs blood flow directly at the target biological tissue(s) or localised compression of biological tissue(s) temporarily impairs blood flow to a defined region or peripheral part of the human or other animal.

Preferably, localised compression of biological tissue(s) temporarily reduces blood volume directly at the target biological tissue(s) or localised compression of biological tissue(s) temporarily reduces blood volume at a defined region or peripheral part of the human or other animal.

Preferably, compression of biological tissue(s) reduces blood volume in the target biological tissue(s).

Preferably, compression improves oximetry/spectroscopy detection.

Preferably, the method comprising analysing the blood volume reduced target biological tissue(s) to determine:
a) skin type and/or skin tone of the target biological tissue(s);
b) one or more molecular constituents of the target biological tissue(s);
c) wavelengths of light having similar properties of optical absorption by the target biological tissue(s) and differential absorption with respect to deoxygenated and oxygenated haemoglobin; and/or
d) one or more wavelengths of light having properties of low absorption by the target biological tissue(s), and high or low absorption with respect to oxygenated or deoxygenated blood;
e) skin type and/or skin tone and adjusting one or more parameters of detection to optimise PPG and/or pulse oximetry detection for a/the specific individual;
f) skin type and/or skin tone and adjusting an/the algorithm output to optimise PPG and/or pulse oximetry detection for a/the specific individual; and/or
g) a baseline measurement for skin colour or pigmentation of an/the individual, and utilising that baseline (preferably to affect a/the algorithm) to improve subsequent detection.

Preferably, analysing the blood volume reduced target biological tissue(s) to determine:
a first wavelength of light having properties of low absorption by the target biological tissue(s), and differential absorption with respect to oxygenated haemoglobin and deoxygenated haemoglobin; and
a second wavelength of light having properties of low absorption by the target biological tissue(s), and:
   a detectable difference with respect to oxygenated haemoglobin and deoxygenated haemoglobin;
   similar absorption with respect to oxygenated haemoglobin and deoxygenated haemoglobin; or
   less or inversely differential absorption characteristics with respect to oxygenated haemoglobin and deoxygenated haemoglobin when compared to absorption of the first wavelength of light.

Preferably, analysing the blood volume reduced target biological tissue(s) to determine: a first wavelength of light having properties of low absorption by the target biological tissue(s), and high absorption with respect to deoxygenated blood; and a second wavelength of light having properties of low absorption by the target biological tissue(s), and low absorption with respect to deoxygenated blood.

Preferably, the method comprising:
adjusting the wavelengths of first and second beams of light according to melanin levels of a specific individual;
adjusting the wavelengths of first and second beams of light according to detected optical reflectance and/or transmission characteristics of the blood volume reduced target biological tissue(s); and/or
adjusting the wavelengths of first and second beams of light to have a detectable differential with respect to oxygenated/deoxygenated haemoglobin, and low, or similar, absorption characteristics relating to the blood volume reduced biological tissue(s).

Preferably, the method comprising:
determining skin type and/or skin tone, and adjusting one or more characteristics of the emitted one or more beams of light to optimise detection for target biological tissue(s) of a/the specific individual;
determining skin type and/or skin tone and adjusting the red, green, and/or blue, and/or infrared amplitudes, or other characteristics, of the emitted one or more beams of light to optimise PPG and/or pulse oximetry detection for a/the specific individual; and/or
determining one or more molecular constituents of the target biological tissue(s) comprises Raman or infrared spectroscopy (or other spectroscopic process) of the target biological tissue(s).

Alternatively, and also preferably, additional biometric testing (for example PPG and/or pulse oximetry detection) may be conducted on biological tissue(s) remotely located from the blood volume reduced target biological tissue(s).

According to a second aspect, the invention provides an apparatus for capturing biometric data of a human and/or other animal, the apparatus comprises:
means for applying localised compression to biological tissue(s) of the human and/or other animal, so as to reduce blood volume in illuminated target biological tissue(s);
means for detecting:
   optical reflectance and/or transmission characteristics of the blood volume reduced, illuminated target biological tissue(s); and/or
   (optical) spectroscopy characteristics of the blood volume reduced, illuminated target biological tissue(s); and
means for analysing biometric data of the means for detecting.

Preferably, the apparatus comprises:
means for illuminating the target biological tissue(s) configured to emit one or more beams of light of one or more wavelengths; and/or
the means for detecting is configured to detect ambient light.

Preferably, the apparatus comprises means for detecting optical reflectance and/or transmission characteristics of non-blood volume reduced target biological tissue(s) and means for analysing biometric data of the means for detecting.

Preferably, the means for detecting and means for analysing conduct detection and analysis on both blood volume reduced and non-blood volume reduced, illuminated target biological tissue(s).

Preferably, the means for detecting and means for analysing optical reflectance and/or transmission characteristics of blood volume reduced and non-blood volume reduced biological tissue(s) is/are configured to conduct detection on the same target biological tissue(s).

Preferably, the means for detecting and means for analysing are located remotely from each other, although operatively connected (for wireless or wired communications).

Preferably, the means for applying localised compression is configured to:
compress biological tissue(s) to reduce or prevent detection of an optical waveform/pulse signal; and/or
compress biological tissue(s) to provides at least a compressive pressure which: reduces or prevents blood flow to target biological tissue(s);
compresses underlying blood vessels; provides a blanching effect to target biological tissue(s); and/or is equivalent to, or exceeds, a systolic blood pressure of an individual.

Preferably, the means for applying localised compression is: one or more (preferably a pair of) inwardly or outwardly opposed compressive surfaces; a tourniquet; a medical cuff (either inflatable or otherwise tightenable); any housing or surface sufficient to transmit compression, a housing or lens of wearable electronics; an inflatable member or housing; or a pressure transmissive surface or member.

Preferably, the apparatus is a sensor apparatus; a smartphone; a patch; an adhesive pad or plaster; a finger clip or pocket; a squeezable pad; a smartwatch; a ring; an inflatable cuff; a wrap-around sensor apparatus; an ear-lobe clip; ear apparatus and/or spectroscopy apparatus, each having at least one means for detecting being a camera, photodiode, PPG sensor, or spectroscopy sensor configured for detecting: optical reflectance and/or transmission characteristics; and/or (optical) spectroscopy characteristics, of the blood volume reduced, illuminated target biological tissue(s).

Preferably, the apparatus is configured to:
analyse one or more differences in the optical reflectance and/or transmission characteristics of the blood volume reduced target biological tissue(s) and non-blood volume reduced target biological tissue(s), and adjust analysis of non-blood volume reduced target biological tissue(s) and/or apply one or more correction factors to analysis of non-blood volume reduced target biological tissue(s); and/or
utilise analysis of the optical reflectance and/or transmission characteristics of the blood volume reduced target biological tissue(s) to improve oximetry, Raman and/or IR spectroscopy, and/or other spectroscopy, of the target biological tissue(s).

Preferably, the apparatus is configured to apply and/or transmit localised compression to:
biological tissue(s) being different tissue(s) to the target biological tissue(s); or
biological tissue(s) being same tissue(s) as the target biological tissue(s).
   Preferably, the blood volume reduced target biological tissue(s) is:
directly compressed biological tissue(s); or
non-compressed biological tissue(s).
   Preferably, the apparatus is configured to:
adjust the wavelengths of first and second beams of light according to melanin levels of a specific individual;
adjust the wavelengths of first and second beams of light according to detected optical reflectance and/or transmission characteristics of the blood volume reduced target biological tissue(s); and/or
adjust the wavelengths of first and second beams of light to have a detectable differential with respect to oxygenated/deoxygenated haemoglobin, and low, or similar, absorption characteristics relating to the blood volume reduced biological tissue(s).
   Preferably, the apparatus is configured to:
determine skin type and/or skin tone, and adjust one or more characteristics of the emitted one or more beams of light to optimise detection for target biological tissue(s) of a/the specific individual;
determine skin type and/or skin tone and adjust the red, green, and/or blue, and/or infrared amplitudes, or other characteristics, of the emitted one or more beams of light to optimise PPG and/or pulse oximetry detection for a/the specific individual;
determine one or more molecular constituents of the target biological tissue(s) comprising Raman or infrared spectroscopy (or other spectroscopic process) of the target biological tissue(s);
determine skin type and/or skin tone and adjust one or more parameters of detection to optimise PPG and/or pulse oximetry detection for a/the specific individual; and/or
determine a baseline measurement for skin colour or pigmentation of an/the individual, and utilising that baseline (preferably to affect a/the algorithm) to improve subsequent detection.

Preferably, the means for analysing comprises a processor and associated algorithm, for analysing biometric data from the means for detecting, so as to determine:
a) skin type and/or skin tone of the illuminated target biological tissue(s);
b) one or more molecular constituents of the illuminated target biological tissue(s);
c) wavelengths of light having similar properties of optical absorption by the illuminated target biological tissue(s) and differential absorption with respect to deoxygenated and oxygenated haemoglobin;
d) one or more wavelengths of light having properties of low absorption by the illuminated target biological tissue(s), and high or low absorption with respect to oxygenated or deoxygenated blood; and/or
e) skin type and/or skin tone and adjust the algorithm output to optimise PPG and/or pulse oximetry detection for a/the specific individual.

According to a third aspect, the invention provides use of an apparatus according to the second aspect for capturing biometric data of a human and/or other animal, the use comprising:
utilising the means for applying localised compression to biological tissue(s) to apply localised compression to the biological tissue(s) and reduce blood volume in target biological tissue(s), and illuminating the target biological tissue(s); OR
illuminating the target biological tissue(s), and utilising the means for applying localised compression to biological tissue(s) to apply localised compression to the biological tissue(s) and reduce blood volume in target biological tissue(s); and
utilising the means for detecting to detect:
   optical reflectance and/or transmission characteristics of the blood volume reduced target biological tissue(s); and/or
   (optical) spectroscopy characteristics of the blood volume reduced target biological tissue(s); and
utilising the means for analysing to analyse biometric data received from the means for detecting.

Preferably, the use comprises any one or more features of the first and/or second aspects.

Advantageously, the present invention reduces the effects of blood, blood volume and blood flow on the optical characteristics of the biological tissue(s) being detected.

Compression of the biological tissue reduces blood flow and, thereby, blood volume within the target biological tissue(s) and, so, reduces the effect(s) of blood flow/volume on detected optical characteristics.

Advantageously, the invention makes peripheral oximetry and spectroscopy racially inclusive, and improves spectroscopy by reducing the potentially adverse-effects of haemoglobin. The method and apparatus reduce the effects of skin colour on such measurements and, thereby improves oximetry and spectroscopy for people with black or brown skin. Further, the present invention improves detection by optimising spectroscopy and oximetry for an individual's skin tone at a/the target tissue(s).

Advantageously, the method and apparatus utilise the detected blood volume reduced characteristics and/or readings to adjust for the melanin effect and/or other pigment changes or variations of an individual's skin (for example caused by jaundice, haemochromatosis, scarring, inflammation, etc.), and adjusts an/the algorithm to optimise detection for target biological tissue(s) of the specific individual.

Advantageously, analysing optical reflectance and/or transmission characteristics of the blood volume reduced target biological tissue(s) (potentially alone) provides a measure of skin colour and/or pigmentation (for example where an individual is suffering from jaundice or haemochromatosis, or exhibits pigmentation changes following treatment, such as with amiodarone).

For the avoidance of doubt, those skilled in the art will understand that increasing or retaining blood volume in target biological tissue(s)—such as an engorged finger—is to be avoided since this provides the opposite of the intended reduction in blood volume.

### BRIEF DESCRIPTION OF THE DRAWING FIGURES

The invention will now be disclosed, by way of example only, with reference to the following drawings, in which:

FIGS. 1a and 1b are graphs showing PPG waveform, having time in the x-axis and wave displacement in the y-axis;

FIG. 2 is a schematic view of a smartphone camera embodiment of the invention;

FIGS. 3a and 3b are schematic views of a PPG monitor embodiment of the invention;

FIG. 4 is a schematic view of a smartwatch embodiment of the invention;

FIG. 5 is a schematic view of a ring embodiment of the invention;

FIGS. 6a and 6b are schematic views of an adhesive pad embodiment of the invention;

FIG. 7 is a schematic view of a wrappable sensor apparatus embodiment of the invention;

FIG. 8 is a schematic view of a first ear sensor apparatus embodiment of the invention;

FIGS. 9a and 9b are schematic views of second and third ear sensor apparatus embodiments of the invention;

FIG. 10 is a graph showing the optical spectrum of melanin, having wavelength on the x-axis and absorption on the y-axis; and

FIGS. 11a and 11b are Raman spectroscopy plots, having wavelength on the x-axis and intensity on the y-axis, showing differential absorption characteristics for whole blood (FIG. 11a) and plasma (FIG. 11b).

### DETAILED DESCRIPTION OF THE INVENTION

The present invention seeks to improve detection of one or more optical reflectance and/or transmission characteristics through temporarily impairing blood flow to target biological tissue(s) and, thereby, reducing blood volume in the target biological tissue(s). In particular, the invention seeks to improve detection and/or analysis of biometric data relating to pulse waveform, oxygen saturation, and/or other optical spectroscopic characteristics of target biological tissue(s).

The invention will first be generally disclosed, before each of the embodiments is disclosed in more detail.

Each embodiment of the present invention generally involves capturing biometric data of a human and/or other animal through applying localised compression to biological tissue(s), so as to reduce blood volume in target biological tissue(s)—which may or may not be the same tissue(s). The target biological tissue(s) is illuminated—with ambient light or light from one or more emitters. The order of illumination or compression is unimportant. Light reflected and/or transmitted through the target biological tissue(s) is detected, and the biological data created is analysed by the processor and its associated algorithm to determine one or more optical reflectance and/or transmission characteristics of the blood volume reduced target biological tissue(s). The one or more optical reflectance and/or transmission characteristics have various uses, which are elaborated upon below. In an alternative, one or more spectroscopy characteristics are determined through detection and analysis following scattering, absorption, emission, and/or reflection of light by the target biological tissue(s).

The degree of compression required to reduce blood volume in target biological tissue(s) depends upon the tissue(s) to be compressed, and also whether it is the same biological tissue(s) as the target tissue(s). Sufficient compression can be determined by loss of the pulse signal/AC (alternating current) nature of the reflected or transmitted light signal. Through extinguishing the AC component and, potentially, in addition the fluctuating venous component, one may derive a baseline reflectance or transmission of the target biological tissue for a detected skin colour. In addition, sufficient compression may be shown by a noticeable change in the colour of the target biological tissue(s), for example a blanching effect on that target biological tissue(s).

Although loss of optical waveform is a measure of impaired blood flow and, therefore, indicative of a reduction in blood volume, one need not actually measure loss of optical waveform in some embodiments. Firm pressure, or pressure above a perceived threshold for the location of the compression—which thereby compresses underlying veins or is above the maximum systolic pressure—may be achieved without needing to prove or detect loss of waveform.

For oximetry, when one or two constant wavelengths are typically utilised, for example red and infrared, a processor and associated algorithm analyses the detected biological signals and controls a baseline red/infrared absorption in non-perfused tissue(s). The output of the algorithm accounts for an expected differential absorption by a higher amount of melanin in pigmented skin, such that relative differences in optical absorption for melanin is specifically taken into consideration for each of the amplitudes of the red/infrared wavelengths detected and, thereby, the processor and algorithm outputs an oximetry determination that is controlled and reliable for the individual's specific skin colour or skin tone.

Target biological tissue(s) with reduced blood volume/blood flow may have optical characteristics determined as a baseline measurement. For example, this enables measurement of any optical or spectroscopic type using a variety of wavelengths, for example using red/green/blue, infrared, or any other visible or non-visible wavelengths. Ambient light, of course, falls within this.

The baseline measurement may be used to: determine skin type (e.g. for considering risk of UV (ultra violet) damage or burning, etc.); determine skin colour/skin tone; provide a baseline for improving analysis, e.g. through the algorithm, of other measures such as spectroscopy and/or oximetry, which reduces any adverse effects of skin colour on accurate detection of biometric data; and/or provides spectrographic information with less effect of dominant haemoglobin spectra and/or other blood component spectra.

In particular, the baseline measurement may be used to improve oximetry algorithms for people with black or brown skin, and controlled for their own individual skin tone at the target biological tissue(s). This allows correction of inaccurate measures based on the ratio of absorption or reflectance of different wavelengths, when absorption and/or reflectance qualities of the target tissue is different between two wavelengths—for example, during pulse oximetry for dark pigmented target skin areas.

With respect to spectroscopy, optical characteristics may differ between different frequencies of incident light, the baseline measurement allows correction for each specific tested wavelength, band of wavelengths, and/or colour.

Determination of the baseline provides the ideal wavelength(s) to be selected for the individual according to the effect of the target skin colour on absorption or reflectance of light wavelengths, so that a wavelength with the least absorption by melanin can be chosen specifically for the individual and specific to the required biological measurement.

With reference to oximetry, oximetry typically requires two wavelength bands whose absorption is differentially affected by oxygenated and/or deoxygenated haemoglobin. Selecting two different wavelengths that have least (and similar) absorption by the melanin of an individual's target biological tissue(s), but provides a detectable (preferably greater) difference in absorption by oxygenated as compared to deoxygenated haemoglobin provides more accurate oxygen saturation measurements—which are specific to the individual's skin colour.

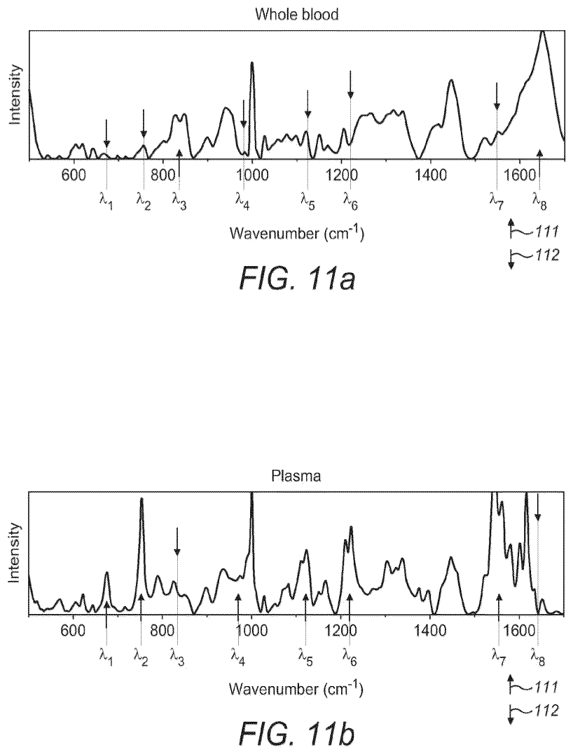

With reference to spectroscopy, including Raman and infrared, target biological tissue(s) is, thereby, less affected by haemoglobin and other blood components, which provides additional information—biometric data—not otherwise readily available. This is elaborated upon with reference to FIGS. 11a and 11b.

A processor and associated electronics, including its associated algorithm, may be located in the same apparatus as the emitter and/or sensor, or may be remotely located. If remotely located, the emitter and/or sensor is/are capable of communicating either wirelessly or through wired communications with an external computer, for analyses of the detected signals and/or recording of data signals.

The algorithm of the processor receives data signals from the sensors and analyses those signals to compare synchronous data on received amplitude and wavelengths of light from single or multiple sensors, or conduct this in an asynchronous manner, for example if the emitters alternate between emitting different wavelengths of light.

In further variants, the emitter may be: a single light source, for example a white light source or light of narrow wavelength; be a multi-spectral light source; or be multiple light sources providing different wavelengths of light. For example, a red light source in addition to an infrared or near-infrared light source.

The sensor/detector may be a single sensor, or multiple sensors, responsive to multiple wavelengths of light, for example a CMOS (complementary metal-oxide-semiconductor) camera sensor sensitive to Red, Green, Blue (RGB), RGB and infrared, or light of narrow wavelength.

The sensors/detectors may include those consisting of arrays, for example CMOS camera sensors, and receive transmitted/reflected light biometric data from the tissue. Such data may be represented graphically by the algorithm as two-dimensional data, images or maps of the tissue. For example, such two-dimensional data, images or maps may be used to define “heat-maps” of relative perfusion and/or oxygenation of target tissues, which may be further represented as one or more individual static images or video images (in real-time or retrospective).

The sensor of the apparatus may be an optical sensor that detects a range of wavelengths of light, which may be achieved by optical filters, gratings, multiple sensors or sensor arrays, or optoelectronics mechanisms, whereby the sensor(s) response to different wavelengths of light is varied (for example tuneable filters including those incorporating a MEMS (Micro-electromechanical systems) Fabry-Perot Interferometer tuneable filter), or camera, which may be of CCD (charge-coupled device) or CMOS sensor type.

By way of an alternative, or in addition, the sensor of the apparatus may be a molecular spectroscopy means comprising at least part of an emitter and associated detector configured to capture biometric data relating to the molecular constituents of blood or tissue of the human or other animal. The spectroscopy means is, preferably, Raman spectroscopy means or infrared spectroscopy means. Accordingly, the spectroscopy characteristics are: Raman scattered light, or infrared absorption, emission, or reflection characteristics. However, other optical spectroscopy may be envisaged.

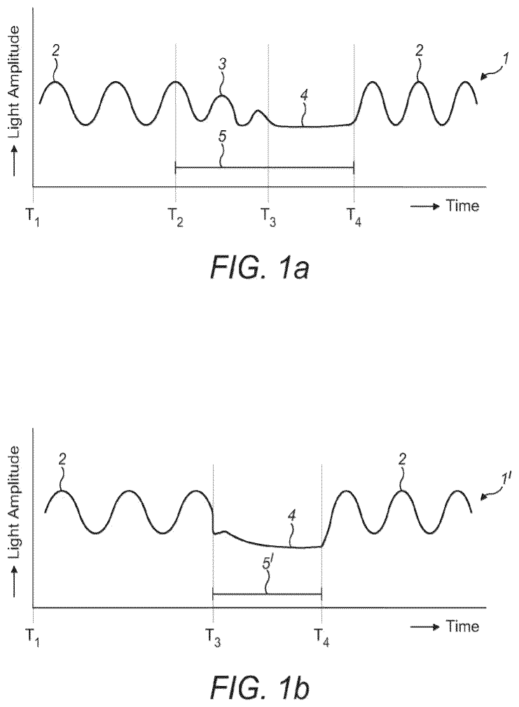

FIG. 1a is relevant to all of the embodiments and shows displacement against time for a detected PPG waveform. Various times T1, T2, T3 and T4, are shown along the x-axis component, and each time defines a change in events during compression of biological tissue(s).

As can be seen from FIGS. 1a-1b, a PPG waveform generally identified by reference 1, is shown to have a regular pulse wave amplitude 2 between time periods T1 and T2. At T2, localised compression is provided to biological tissue(s) of the individual so as to impair blood flow to target biological tissue(s) and, thereby, reduce blood flow to and blood volume in the target biological tissue(s), and between time periods T2 and T3 the pulse wave amplitude 3 decreases to zero, shown at T3. At T3, those skilled in the art will understand that sufficient compressive force has been applied to the biological tissue(s) so as to prevent detection of an optical waveform/pulse signal from the target biological tissue(s).

Between time periods T3 and T4, localised compression is maintained and the target biological tissue(s) has significantly reduced blood volume, and blood flow is substantially prevented, which maintains the pulse wave amplitude 4 at zero. In the claims this tissue is defined as ‘blood volume reduced target biological tissue(s)’. At time period T4, localised compression is removed and the PPG waveform returns to its normal amplitude 2, having similar pulse wave amplitude 2 and pulse waveform as between time periods T1 and T2. In the claims, this tissue before and after compression is defined as ‘non-blood volume reduced target biological tissue(s)’.

FIG. 1b shows a variant in which a calibration step is included during detection of the biometric data. The effect of the localised compression 5′ is near instantaneous, and there is no time period T2 where compression increases gradually and amplitude decreases gradually. Between T1 and T3 the PPG waveform 1′ is shown to have a regular amplitude 2. Calibration occurs during a period of localised compression 5′, between time periods T3 and T4 (during which localised compression is maintained). A calibration tone or signal may be used to inform the individual at or just before T4 when calibration has completed and localised compression 5′ can be removed. A further measurement of biometric data could be collected during periods of regular amplitude 2, being either before or after calibration, save that, whether before or after, calibration is used to improve PPG analysis of the non-blood volume reduced biological tissue(s).

In each of the following embodiments, detection and analysis of the optical reflectance and/or transmission characteristics of the blood volume reduced target biological tissue(s) is performed with a view to improving the capture of other biometric data. In particular, one can, thereby, improve detection conducted on non-blood volume reduced biological tissue(s).

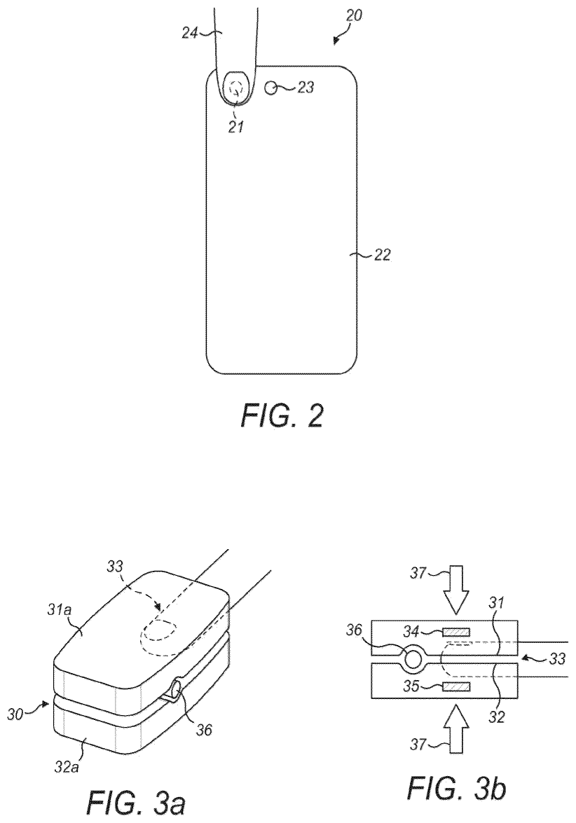

FIG. 2 shows a smartphone, generally identified by reference 20. A camera 21 is located on the outside of a housing 22 of the smartphone 20, as is well known in the art. The camera 21 is capable of detecting multi-spectral light. A light 23, or LED (light emitting diode) 23, is located in proximity to the camera 21, so as to provide illumination of target biological tissue(s), although this is not essential as illumination could be ambient light. In this embodiment, the target biological tissue(s) is a fingertip 24 of an individual.

In use, the fingertip 24 is held against the camera 21, and either ambient light or light from the adjacent light 23 transmits through the fingertip 24 or is reflected from a surface of the fingertip 24, and detected by the camera 21. Through holding or resting his/her fingertip 24 against the camera, this allows a PPG waveform to be detected—although this could take place after compression. The individual presses more firmly his/her fingertip 24 against the camera 21 to a compressive force where the PPG waveform is extinguished and blood volume reduced. One or more optical characteristics are determined from analysis of the biometric data. An algorithm of a processor analyses the red/green/blue image intensities when there is no PPG waveform/reduced blood volume, and applies a correction factor to the red/green/blue amplitudes captured during normal PPG waveform detection, which may occur subsequently to reduction or removal of the compressive force (to restore the PPG waveform), or before such force is applied (as mentioned above). Calculation of blood oxygen saturation—which, for example, may include the standard ‘ratio of ratios’—is, therefore, corrected for underlying skin colour on the individual, providing individualised self-correction for the target site—biological tissue(s)—being analysed.

By way of an alternative, the camera may be a different sensor, for example a photodiode or video camera.

FIGS. 3a-3b are an embodiment based upon a standard fingertip pulse oximeter, identified generally by reference 30, and adapted according to the present invention. The oximeter 30 includes two opposed surfaces 31; 32, for receipt of a fingertip 33 of an individual, and further includes an emitter 34 and sensor 35. The emitter 34 is one or more LEDs 34, for emitting red and infrared light, which light is detectable by a photodiode, being the sensor 35. The emitter 34 is housed in a first part 31a of the oximeter 30 having surface 31 and the detector 35 is housed in a second part 32a of the oximeter 30 having surface 32, with each surface 31; 32 inwardly facing towards where the fingertip 33 is locatable. The two opposed surfaces 31; 32 are biased inwardly about a pivot point 36, allowing the oximeter 30 to be opened for receipt of the fingertip 33 and also to grip the fingertip 33 in use; however, as a standard fingertip pulse oximeter 30 requires blood volume and/or blood flow in the finger to detect pulse waveform and oximetry, it is not designed to overly compress the fingertip 33.

The invention, therefore, may involve modifying and increasing the biasing force of the pivot point 36 or providing additional compressive force 37, which may be provided by pressing together or squeezing the two surfaces 31; 32 around the fingertip 33. Further the oximeter may be provided with automatic compression, in the form of a cuff or inflatable portion(s) for providing a compressive force which extinguishes PPG waveform and, thereby, reduces blood volume in the fingertip 33.

Although the pulse oximeter is referred to as a fingertip pulse oximeter, it will, of course, be understood by those skilled in the art that the such pulse oximeters may with little if any adaption be used on an ear, toe, wrist, sternum or other region of skin of an individual.

Although absorption is measured in the variant above, reflectance may form the basis of detection where the oximeter has both the emitter and detector on the same inwardly facing surface.

In use, surfaces 31; 32 are separated against the biasing force of the pivot point 36, and the fingertip 33 of the individual is placed between surfaces 31; 32, where it is gently gripped. An increased compressive force 37 is provided by squeezing parts 31a and 32a together to, thereby, compress the fingertip 33 and prevent blood flow and expel blood from the fingertip 33. The increased compressive force 37 may be applied by the individual's other hand or by a health practitioner (for example). In this state, the PPG waveform is extinguished and blood volume reduced in the fingertip 33, and red and infrared light are subsequently emitted by the emitter 34 and separately detected by the detector 35, and the biometric data is analysed. Further detection takes place once the increased compressive force 37 has been removed—although the order could be reversed.

An algorithm of a processor analyses the red/infrared intensities when there is no PPG waveform/reduced blood volume, and applies a correction factor to the red/infrared amplitudes captured during PPG waveform detection, which may occur subsequently to reduction or removal of the compressive force 37, or before such force is applied.

Accordingly, calculation of oxygen saturation is, therefore, corrected for underlying skin colour of the individual, providing individualised self-correction for the target tissue being analysed.

An individual (or third party) may press on the sensor or squeeze the oximeter until a calibration tone or signal is received, confirming calibration has completed. The compressive force may then be reduced or removed to provide detection of normal PPG waveform, analysis of which is improved through use of one or more correction factors determined during detection of blood volume reduced biological tissue(s).

In a variant, a cuff may be inflated to extinguish the PPG waveform for obtaining that measurement, and then deflated to obtain a corrected (or modified) measurement of PPG waveform, without removing the oximeter. The cuff would be intended to exert direct pressure on target biological tissue which, for the purposes of this example, would mean direct pressure to compress the fingertip 33 and prevent blood flow and expel blood from the fingertip 33.

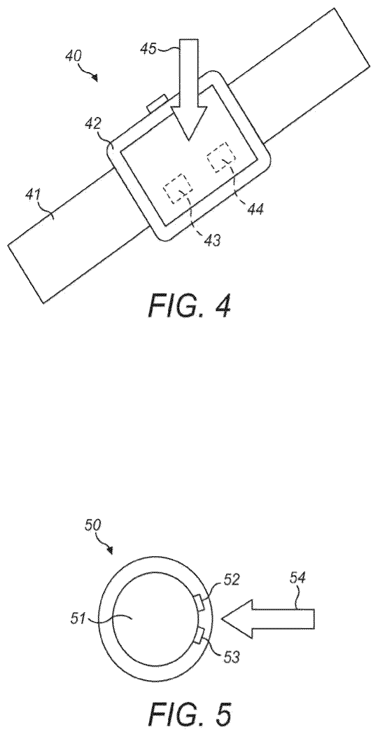

FIG. 4 shows a smart watch, identified generally by reference 40. The smartwatch 40 includes a strap 41 and watch face 42, as is standard; however, further includes an emitter 43 and a sensor 44 on an underside of the watch face 42. The emitter 43 and sensor 44 are located so as to face an arm/wrist region (not shown) of an individual and configured to capture biometric data of the individual.

The emitter 43 is one or more LEDs 34, for emitting red and infrared light, which light is detectable by a photodiode, being the sensor 44. The strap 41 is tightenable around the arm/wrist region of the individual, in use, which brings the emitter 43 and sensor 44 into proximity (or preferably contact) with the individual. However, the strap 41 is not designed to overly compress the arm. It must be comfortable and not restrict blood flow during normal use/activities.

In use of the invention, an increased compressive force 45 is applied to press the underside of the watch face 42 firmly onto the arm/wrist region and, thereby, compress skin under the underside of the watch face 42, and prevent blood flow and expel blood from that skin. The increased compressive force 45 may be applied by the individual's other hand or, potentially, a third party. In this state, the PPG waveform is extinguished and blood volume to that skin reduced, and red and infrared light are separately emitted by the emitter 43 and separately detected by the sensor 44, and the biometric data is analysed. Further detection takes place once the increased compressive force 45 has been removed—or potentially first, before it is applied.

Analysis and/or calibration is in-line with that described above for FIGS. 3a-3b, such that calculation of oxygen saturation is, therefore, corrected for underlying skin colour of the individual, providing individualised self-correction for the target site being analysed.

FIG. 5 shows a ring, identified generally by reference 50. The ring 50 includes a typical aperture 51 for receipt of a finger (not shown) of an individual, as is standard; however, further includes an emitter 52 and a sensor 53 on an underside of the ring 50. The emitter 52 and sensor 53 are located so as to face the finger of the individual, when worn, and are configured to capture biometric data of the individual. The emitter 52 is one or more LEDs 52, for emitting red and infrared light, which light is detectable by a photodiode, being the sensor 53. The ring 50 is intended to be worn by an individual, which brings the emitter 52 and sensor 53 into contact with the individual; however, the ring 50 is not designed to overly compress the finger as this would cut-off blood supply to the finger. It must be comfortable and not restrict blood flow during normal use/activities.

In use of the invention, an increased compressive force 54 is applied to press the ring 50 onto the finger of the individual more firmly and, thereby, compress skin under the ring 50, and prevent blood flow and expel blood from that skin. The increased compressive force 54 may be applied by the individual's other hand or, potentially, a third party. In this state, the PPG waveform is extinguished and blood volume to that skin reduced, and red and infrared light are separately emitted by the emitter 52 and separately detected by the sensor 53, and the biometric data is analysed. Further detection takes place once the increased compressive force 54 has been removed—or potentially first, before it is applied.

Analysis is in-line with that described above for FIGS. 3a-3b and 4 such that calculation of oxygen saturation is, therefore, corrected for underlying skin colour of the individual, providing individualised self-correction for the target tissue being analysed.

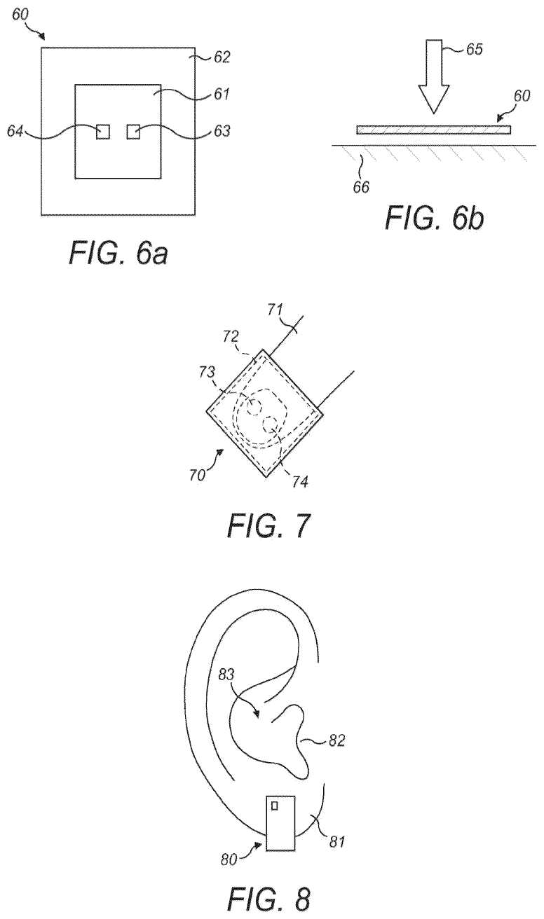

FIG. 6a shows an underside of an adhesive pad, identified generally by reference 60. The pad 60 includes a central region 61, surrounding which is an adhesive region 62. The central region 61 includes an emitter 63 and a sensor 64 on what will be, in use, an underside or skin-facing side of the pad 60—being also the side to which adhesive is located in the adhesive region 62. Accordingly, the emitter 63 and sensor 64 are located so as to face the skin of the individual at the site of application of the adhesive pad 60, and are configured to capture biometric data of the individual.

In use, as shown in FIG. 6b, the adhesive pad 60 is applied to the skin 66 of the individual in a target region, an increased compressive force 65 is applied to press the pad 60 onto the skin of the individual and, thereby, compress skin under the pad 60, and prevent blood flow and expel blood from that skin. The increased compressive force 65 may be applied by the individual's hand or, potentially, a third party. In this state, the PPG waveform is extinguished and blood volume in the target region reduced, and red and infrared light are separately emitted by the emitter 63 and separately detected by the sensor 64, and the biometric data is analysed. Further detection takes place once the increased compressive force 65 has been removed—or potentially first, before it is applied.

Analysis is in-line with that described above for FIGS. 3a-3b to 5 such that calculation of oxygen saturation is, therefore, corrected for underlying skin colour of the individual, providing individualised self-correction for the target site being analysed.

FIG. 7 shows a wrappable sensor apparatus, identified generally by reference 70. The apparatus 70 is intended to be located around a finger 71 of an individual, although, if sized appropriately, may be used around an arm or leg of the individual. The apparatus 70 is elongate and intended to be wrapped around the finger 71, in use, in a similar way to a blood pressure cuff. Accordingly, it has a hook and loop fastener (not shown) for looping and securing the apparatus 70 around the individual's finger 71. The apparatus 70 includes an inflatable member 72 for applying compression to underlying skin in that region. The inflatable member 72 has a width the same or similar to the apparatus 70 providing compression to underlying skin across substantially the whole width of the apparatus 70; however, that is not essential. The inflatable member 72 provides a configurable pressure effect, allowing an operator, medical practitioner and/or an algorithm of a/the processor to configure how much to inflate the member 72 and, thereby, how much pressure to apply to the finger 71. The apparatus 70 includes an emitter 73 and a sensor 74 on what will be, in use, an inwardly facing surface thereof or skin-facing surface. Accordingly, the emitter 73 and sensor 74 are located so as to face the skin of the individual at the site of application of the apparatus 70, i.e. where underlying skin will be compressed, and are configured to capture biometric data of the individual. Depending upon the locations of the emitter 73 and the sensor 74 with respect to the finger 71, the apparatus 70 may use reflectance or transmission of light.

By way of an alternative, wrapping of the apparatus 70 itself around a user's finger may provide sufficient compression without the need for an inflatable member—although the latter is preferred.

By way of a further alternative, the hook and loop fastener may be replaced by other fastener means.

In use, the apparatus 70 is wound around the finger 71, so as to fully surround the finger 71, and secured. A PPG measurement may be taken prior to applying a compressive force; however, it is preferable to inflate the cuff 72 to apply a compressive force circumferentially around the finger 71 and, thereby, compress skin under the apparatus 70, and prevent blood flow and expel blood from that skin. In this state, the PPG waveform is extinguished and blood volume reduced, and red and infrared light are separately emitted by the emitter 73 and separately detected by the sensor 74, and the biometric data is analysed. Further detection preferably takes place once the inflatable cuff 72 has been deflated.

Analysis is in-line with previous embodiments such that calculation of oxygen saturation is, therefore, corrected for underlying skin colour of the individual.

FIG. 8 shows a first ear sensor apparatus, identified generally by reference 80. The apparatus 80 is an ear-lobe clip, intended to be located, in use, directly on an ear-lobe 81 of an individual, where reflectance or transmission may be used to obtain biometric data. Apart from being sized for an ear-lobe 81 rather than a fingertip, and having relatively flat inwardly facing surfaces, the ear-lobe clip 80 has a configuration and use very similar to the fingertip pulse oximeter of FIGS. 3a and 3b and, so, shall not be described further.

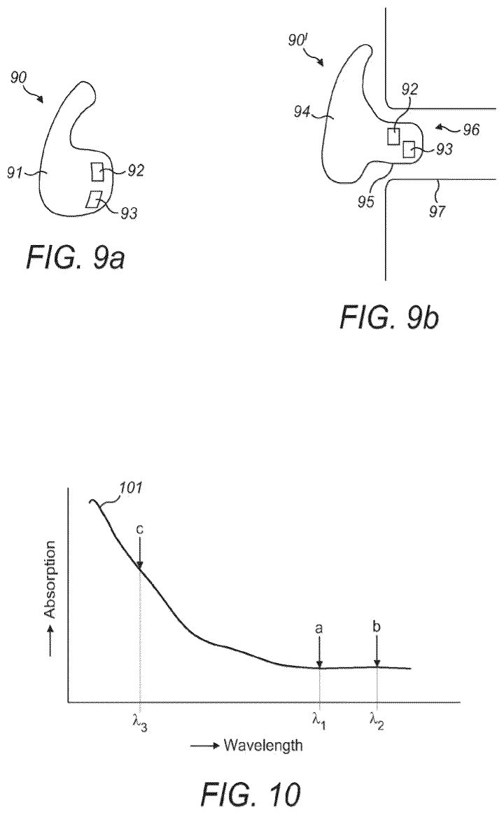

FIGS. 9a and 9b show second and third ear sensor apparatus, identified generally by references 90; 90′.

The first apparatus 90 includes a housing 91 dimensioned and shaped to be received in a concha region 83 (shown in FIG. 8) of an individual's ear, and the housing includes an emitter 92 and a sensor 93, directed to the tragus 82 (shown in FIG. 8) and/or other part of the wall of the concha region 83. Accordingly, the emitter 92 and sensor 93 are located so as to face the skin of the individual's ear, and are configured to capture biometric data of the individual.

The second apparatus 90′ includes a housing 94 having an ear-canal portion 95, for receipt within an ear-canal 96 of the individual. The ear-canal portion 95 includes an emitter 92 and a sensor 93, directed to the ear-canal wall 97. Accordingly, the emitter 92 and sensor 93 are located so as to face the skin of the individual's ear, and are configured to capture biometric data of the individual.

In use, both apparatus 90: 90′ are used similarly, and are appropriately located in the ear of the individual. A PPG measurement may be taken prior to applying a compressive force; however, it is preferable that the individual uses his/her finger (not shown) to push the emitter and sensor 92; 93 towards adjacent skin, and increase such force so as to apply a compressive force onto that adjacent skin, and prevent blood flow and expel blood from that skin. By way of a variant, a third party may apply the compressive force. In this state, the PPG waveform is extinguished and red and infrared light are separately emitted by the emitter 92 and separately detected by the sensor 93, and the biometric data is analysed. Further detection preferably takes place once the compressive force has been removed.

Analysis is in-line with previous embodiments such that calculation of oxygen saturation is, therefore, corrected for underlying skin colour of the individual.

Although FIGS. 2 to 9b are directed predominantly to a disclosure relating to PPG waveform/oximetry detection, different sensors may be used for detection of other biometric data without diverging from the teaching of the invention to obtain measurements from blood volume reduced target biological tissue(s) using localised compression.

The following may be applicable to each embodiment above, as an add-on or an alternative.

FIG. 10 shows the optical spectrum of melanin. Those skilled in the art will understand from FIG. 10 that melanin more readily absorbs light of a shorter wavelength, and absorbs comparatively less light of longer wavelengths.

FIG. 10 relates to an embodiment in which an emitter is capable of providing multi-spectrum light at a time the biological tissues is compressed, and a sensor is capable of detecting that multi-spectrum light. Each of the apparatus in FIGS. 2 to 9b could be modified and/or adapted accordingly.

The processor and its associated algorithm are capable of analysing the detected multi-spectrum light to ascertain the absorption properties of melanin in an individual's skin, and determine a range of wavelengths of light which exhibit low absorption by the individual's skin.

FIG. 10 shows a plot of amplitude of absorption against wavelength being an arcuate line 101, and provides three exemplary amplitudes: a, b and c, at different wavelengths: λ1, λ2, and λ3. One can see that, at wavelengths λ1 and λ2, absorption a and b is relatively low, but at λ3, absorption is comparatively high—and clearly different to absorption at λ1 and λ2. Accordingly, in determining which wavelengths to use for PPG detection after compression has been removed from the biological tissue(s), one would be motivated to use wavelengths λ1 and λ2, and not λ3.

Advantageously, those wavelengths exhibiting either least absorption by the target tissue(s), or equivalent absorption, are chosen as the wavelengths to be used to detect biological data from uncompressed tissue(s), and such functionality may be included as an add-on or as an alternative in the embodiments of FIGS. 2 to 9b.

The following may be applicable to one or more embodiments above, as an add-on or an alternative.

FIGS. 11a-11b shows Raman spectroscopy graphs exemplifying the differential absorption characteristics of whole blood (FIG. 11a), containing blood cells including red blood cells, as compared to plasma (FIG. 11b), being devoid of blood cells. Accordingly, through this approach, one can compare absorption characteristics of skin substantially minus any effects of haemoglobin absorption.

Whilst it is not intended to be bound by theory, it is understood that haemoglobin avidly absorbs certain wavelengths of light, but reflects red light—being part of the theory upon which standard PPG detection is based. It is further understood that the spectroscopic signals from haemoglobin may saturate signals from other molecules. Through reducing the amount of blood in target biological tissue(s)—and, thereby, the haemoglobin—by compression of biological tissue(s), scattered light can be detected; however the biometric data will then be provided in a normalised manner, with reduced, or without, any adverse effects of haemoglobin absorption—which is, effectively, removed from the biometric data.

One can see from FIGS. 11a and 11b that absorption characteristics of whole blood as compared to plasma are different at various wavelengths λ1 to λ8, where arrows have been included to indicate which spectrum has the greater relative amplitude (an up arrow 111) and which the lower amplitude (a down arrow 112).

One can, therefore, improve Raman, infrared and of other spectroscopic processes by compressing biological tissue(s), extinguishing the PPG waveform and reducing blood volume from target biological tissue(s) before conducting spectroscopic analysis. Such functionality may be included as an add-on or as an alternative in the embodiments of FIGS. 2 to 9b.

Although embodiments relating to FIGS. 2 to 9b describe apparatus and methods for detection of PPG waveform, these are examples and the emitters and/or sensors of these embodiments may be differently configured to detect other biometric data—for example spectroscopic data.

In a further variant, the invention may include stand-off detection, for remotely capturing biometric data from a human or other animal. This may further include: (means for) controlling a distance, position and/or location of one or more sensor means and/or emitter with respect to remotely located target biological tissue(s); and/or (means for) controlling a direction and/or point of focus of emitted light with respect to remotely located target biological tissue(s).

Those skilled in the art will understand that, in some embodiments, the target biological tissue(s) may be directly compressed and analysed and, in some others, the biological tissue which is compressed is not necessarily the same as the target biological tissue(s). Those skilled in the art will also realise that calibration, for the purpose of detecting skin type and/or skin tone so as to optimise further biometric testing for a/the specific individual, and involving compression of target biological tissue(s), may be conducted on different and remotely located tissue to the site of that further biometric testing.

1.-24. (canceled)

25. A method for capturing biometric data of a human and/or other animal, the method comprising:
(a) either (i) applying localized compression of biological tissue(s), so as to reduce blood volume in target biological tissue(s), and illuminating the target biological tissue(s); or (ii) illuminating the target biological tissue(s) and applying localized compression of biological tissue(s), so as to reduce blood volume in target biological tissue(s); and
(b) detecting and analyzing:
(i) one or more optical reflectance and/or transmission characteristics of the blood volume reduced target biological tissue(s); and/or
(ii) one or more spectroscopy characteristics of the blood volume reduced target biological tissue(s).

26. The method as claimed in claim 25 comprising detecting and analyzing optical reflectance and/or transmission characteristics of non-blood volume reduced target biological tissue(s) either prior to applying localized compression or following reducing localized compression.

27. The method as claimed in claim 25 further comprising reducing localized compression of biological tissue(s) to restore blood flow to target biological tissue(s), and detecting and analyzing optical reflectance and/or transmission characteristics of the non-blood volume reduced target biological tissue(s).

28. The method as claimed in claim 25 comprising detecting and analyzing optical reflectance and/or transmission characteristics of blood volume reduced and non-blood volume reduced biological tissue(s) being conducted on the same target biological tissue(s).

29. The method as claimed in claim 25, wherein illuminating comprises:
illuminating using ambient light; and/or
emitting one or more beams of light of one or more wavelengths.

30. The method as claimed in claim 25, wherein the method comprising:
(i) analyzing one or more differences in the optical reflectance and/or transmission characteristics of the blood volume reduced target biological tissue(s) and non-blood volume reduced target biological tissue(s), adjusting analysis of non-blood volume reduced target biological tissue(s), and/or applying one or more correction factors to analysis of non-blood volume reduced target biological tissue(s); and/or
(ii) utilizing analysis of the optical reflectance and/or transmission characteristics of the blood volume reduced target biological tissue(s) to improve oximetry, Raman and/or IR spectroscopy of the target biological tissue(s).

31. The method as claimed in claim 25 comprising applying localized compression to:
biological tissue(s) being different tissue(s) than the target biological tissue(s); or
biological tissue(s) being the same tissue(s) as the target biological tissue(s).

32. The method as claimed in claim 25, wherein the blood volume reduced target biological tissue(s) is:
directly compressed biological tissue(s); or
non-compressed biological tissue(s).

33. The method as claimed in claim 25, wherein the method comprising analyzing the blood volume reduced target biological tissue(s) to determine any one or more of the following comprising:
a) skin type and/or skin tone of the target biological tissue(s);
b) one or more molecular constituents of the target biological tissue(s);
c) wavelengths of light having similar properties of optical absorption by the target biological tissue(s) and differential absorption with respect to deoxygenated and oxygenated hemoglobin;
d) one or more wavelengths of light having properties of low absorption by the target biological tissue(s), and high or low absorption with respect to oxygenated or deoxygenated blood;
e) the skin type and/or skin tone and adjusting one or more parameters of detection to optimize photoplethysmography (PPG) and/or pulse oximetry detection for a specific individual;
f) skin type and/or skin tone and adjusting an algorithm output to optimize PPG and/or pulse oximetry detection for the specific individual; and/or
g) a baseline measurement for skin color or pigmentation of the specific individual, and utilizing that baseline measurement to improve subsequent detection.

34. The method as claimed in claim 25, wherein the method comprising analyzing the blood volume reduced target biological tissue(s) to determine:
a first wavelength of light having properties of low absorption by the target biological tissue(s), and differential absorption with respect to oxygenated hemoglobin and deoxygenated hemoglobin;
a second wavelength of light having properties of low absorption by the target biological tissue(s); and
either (i) a detectable difference with respect to oxygenated hemoglobin and deoxygenated hemoglobin; (ii) similar absorption with respect to oxygenated hemoglobin and deoxygenated hemoglobin; or (iii) less or inversely differential absorption characteristics with respect to oxygenated hemoglobin and deoxygenated hemoglobin when compared to absorption of the first wavelength of light.

35. The method as claimed in claim 25, wherein illuminating comprises emitting one or more beams of light of one or more wavelengths, the method further comprising:
adjusting the wavelengths of first and second beams of light according to melanin levels of a specific individual;
adjusting the wavelengths of first and second beams of light according to detected optical reflectance and/or transmission characteristics of the blood volume reduced target biological tissue(s); and/or
adjusting the wavelengths of first and second beams of light to have a detectable differential with respect to oxygenated/deoxygenated hemoglobin, and low, or similar, absorption characteristics relating to the blood volume reduced biological tissue(s).

36. The method as claimed in claim 25, wherein illuminating comprises emitting one or more beams of light of one or more wavelengths, the method further comprising:
determining skin type and/or skin tone, and adjusting one or more characteristics of the emitted one or more beams of light to optimize detection for target biological tissue(s) of a specific individual; and/or
determining the skin type and/or skin tone and adjusting red, green, and/or blue, and/or infrared amplitudes, or other characteristics, of the emitted one or more beams of light to optimize PPG and/or pulse oximetry detection for the specific individual.

37. The method as claimed in claim 25, wherein illuminating comprises emitting one or more beams of light of one or more wavelengths, the method further comprising determining one or more molecular constituents of the target biological tissue(s) comprises Raman or infrared spectroscopy of the target biological tissue(s).

38. An apparatus for capturing biometric data of a human and/or other animal, the apparatus comprising:
a compression device for applying localized compression to biological tissue(s) of the human and/or other animal, so as to reduce blood volume in illuminated target biological tissue(s);
a detector for detecting (i) optical reflectance and/or transmission characteristics of the blood volume reduced, illuminated target biological tissue(s), and/or (ii) spectroscopy characteristics of the blood volume reduced, illuminated target biological tissue(s); and
a processor for analyzing biometric data of the detector.

39. The apparatus as claimed in claim 38, wherein the apparatus comprises:
an illumination device for illuminating the target biological tissue(s), said illumination device being configured to emit one or more beams of light of one or more wavelengths.

40. The apparatus as claimed in claim 38, wherein the apparatus comprises a detector for detecting optical reflectance and/or transmission characteristics of non-blood volume reduced target biological tissue(s) and a processor for analyzing biometric data of the detector.

41. The apparatus as claimed in claim 38, wherein the detector and the processor are operatively connected and located remotely from each other.

42. The apparatus as claimed in claim 38, wherein the compression device is configured to:
(a) compress biological tissue(s) to reduce or prevent detection of an optical waveform/pulse signal; and/or
(b) compress biological tissue(s) to provide at least a compressive pressure which:
(i) reduces or prevents blood flow to target biological tissue(s);
(ii) compresses biological tissue(s) to provide a localized blanching effect on the target biological tissue;
(iii) compresses underlying blood vessels; and/or
(iv) provides a compressive pressure equivalent to, or exceeding, a systolic blood pressure of an individual.

43. The apparatus as claimed in claim 38, wherein the compression device is: one or more inwardly or outwardly opposed compressive surfaces; a tourniquet; a medical cuff; any housing or surface sufficient to transmit compression; a housing or lens of wearable electronics; an inflatable member or housing; or a pressure transmissive surface or member.

44. The apparatus as claimed in claim 38, wherein the apparatus is a sensor apparatus; a smartphone; a patch; an adhesive pad or plaster; a finger clip or pocket; a squeezable pad; a smartwatch; a ring; an inflatable cuff, a wrap-around sensor apparatus, an ear-lobe clip, ear apparatus and/or spectroscopy apparatus, each having at least one detector being a camera, photodiode, PPG sensor, or spectroscopy sensor configured for detecting: (i) optical reflectance and/or transmission characteristics; and/or (ii) spectroscopy characteristics, of the blood volume reduced, illuminated target biological tissue(s).

45. The apparatus as claimed in claim 38, wherein the apparatus is configured to:
analyze one or more differences in the optical reflectance and/or transmission characteristics of the blood volume reduced target biological tissue(s) and non-blood volume reduced target biological tissue(s), and adjust analysis of the non-blood volume reduced target biological tissue(s) and/or apply one or more correction factors to analysis of the non-blood volume reduced target biological tissue(s); and/or
utilize analysis of the optical reflectance and/or transmission characteristics of the blood volume reduced target biological tissue(s) to improve oximetry, Raman and/or IR spectroscopy, and/or other spectroscopy of the target biological tissue(s).

46. The apparatus as claimed in claim 38, wherein the apparatus is configured to apply and/or transmit localized compression to:
biological tissue(s) being different tissue(s) than the target biological tissue(s); or
biological tissue(s) being the same tissue(s) as the target biological tissue(s).

47. The apparatus as claimed in claim 38, wherein the apparatus comprises an illumination device configured to emit one or more beams of light of one or more wavelengths, wherein the apparatus is configured to:
determine skin type and/or skin tone, and adjust one or more characteristics of the emitted one or more beams of light to optimize detection for target biological tissue(s) of a specific individual;
determine the skin type and/or skin tone and adjust red, green, and/or blue, and/or infrared amplitudes, or other characteristics, of the emitted one or more beams of light to optimize PPG and/or pulse oximetry detection for the specific individual.

48. The apparatus as claimed in claim 38, configured to:
determine one or more molecular constituents of the target biological tissue(s) comprising Raman or infrared spectroscopy of the target biological tissue(s);
determine skin type and/or skin tone and adjust one or more parameters of detection to optimize PPG and/or pulse oximetry detection for a specific individual; and/or
determine a baseline measurement for skin color or pigmentation of a baseline individual, and utilize that baseline measurement to improve subsequent detection.

49. The apparatus as claimed in claim 38, wherein the processor comprises an associated algorithm for analyzing biometric data from the detector so as to determine:
a) skin type and/or skin tone of the illuminated target biological tissue(s);
b) one or more molecular constituents of the illuminated target biological tissue(s);
c) wavelengths of light having similar properties of optical absorption by the illuminated target biological tissue(s) and differential absorption with respect to deoxygenated and oxygenated hemoglobin;
d) one or more wavelengths of light having properties of low absorption by the illuminated target biological tissue(s), and high or low absorption with respect to oxygenated or deoxygenated blood; and/or
e) the skin type and/or skin tone and adjust an algorithm output to optimize PPG and/or pulse oximetry detection for a specific individual.

50. A method for capturing biometric data of a human and/or other animal with an apparatus, the apparatus including (a) a compression device for applying localized compression to biological tissue(s) of the human and/or other animal, so as to reduce blood volume in illuminated target biological tissue(s); (b) a detector for detecting (i) optical reflectance and/or transmission characteristics of the blood volume reduced, illuminated target biological tissue(s), and/or (ii) spectroscopy characteristics of the blood volume reduced, illuminated target biological tissue(s); and (c) a processor for analyzing biometric data of the detector, the method comprising:
(1) either (i) utilizing the compression device to apply localized compression to biological tissue(s) and reduce blood volume in target biological tissue(s), and illuminating the target biological tissue(s); or (ii) illuminating the target biological tissue(s), and utilizing the compression device to apply localized compression to the biological tissue(s) and reduce blood volume in target biological tissue(s);
(2) utilizing the detector to detect: (i) optical reflectance and/or transmission characteristics of the blood volume reduced target biological tissue(s); and/or (ii) spectroscopy characteristics of the blood volume reduced target biological tissue(s); and
(3) utilizing the processor to analyze biometric data received from the detector.
